(19) United States
(12) Patent Application Publication
(10) Pub. No.: US 2018/0365922 A1
(43) Pub. Date: Dec. 20, 2018

(54) Chalk Vending Machine

(71) Applicant: Limestone Climbing Inc., Shawinigan (CA)

(72) Inventors: Yves-Antoine CRÊTE, Montreal (CA); Guillaume Robitaille BEAUMIER, Verdun (CA)

(73) Assignee: Limestone Climbing Inc., Shawinigan (CA)

(21) Appl. No.: 16/012,282

(22) Filed: Jun. 19, 2018

Related U.S. Application Data
(60) Provisional application No. 62/521,752, filed on Jun. 19, 2017.

(51) Int. Cl. G07F 13/02; A61K 8/02

(52) U.S. Cl. CPC G07F 13/025; A61K 8/0225

(57) ABSTRACT

A chalk vending machine or apparatus and a method of vending chalk are provided. A reservoir is used to store the chalk. A feeder is used to transfer the chalk from the reservoir to the customer. A payment system allows the customer to pay for the product. A mixer is added to prevent the chalk from self-compacting. Motion transfer and generation devices are used to actuate the feeder. Accessories such as a door and a window for the reservoir and a chute and tray for the feeder are added to enhance the usability of the apparatus.

This application claims the benefit of U.S. Provisional Application No. 62/521,752 filed on Jun. 19, 2017, the contents of which are incorporated herein by reference.

### TECHNICAL FIELD

The subject invention relates to vending apparatuses generally, and in particular to a vending apparatuses suitable for dispensing moisture removal agents.

### BACKGROUND ART

A moisture removal agent, referred to herein as “chalk”, is often used in sports to remove body moisture in athletes, particularly in sports where hand grip is an important contributor in athletic performance.

Body moisture removal agents such as chalk are typically distributed and sold to customers in disposable bags. The process of manually transferring the product from a disposable bag to a smaller waist bag or other type of bag or container is often messy and wasteful, some of the chalk typically ending up on the floor during the transfer process.

For distributors, selling chalk on store shelves in disposable bags requires re-packaging the material in individual bags. There are several disadvantages associated with pre-packaging the material. Obviously, it adds to the cost of operations of the business that vends chalk to customers. Further, customers are deprived of the flexibility to buy the exact quantity that meets their needs. As a result, customers often buy more chalk than they actually need, and thus will have to find storage for the extra amount, potentially over a long period.

Chalk is a non-homogeneous material often having irregular granularity. Such materials are known to be subject to self-compaction inside reservoirs, which makes them difficult to distribute in existing vending machines.

There is thus a need for an apparatus that mitigates at least some of the aforementioned disadvantages associated with current conventional methods and apparatuses for vending chalk.

### SUMMARY OF INVENTION

In accordance with one aspect of the present invention, there is provided a vending apparatus that includes a reservoir for storing powder having an opening for dispensing the powder; a mixer disposed within said reservoir; a motion generation device in mechanical communication with the mixer; and a controller for selecting a specific volume of the powder to be dispensed. The controller is operable to selectively cause the motion generation device to rotate the mixer thereby mixing the powder. The controller is also operable to cause the specific volume of powder to be dispensed though the opening. The opening can be connected to a chute. The apparatus may be housed in a housing having a window that allows a user of the apparatus to inspect the powder or chalk level in the reservoir.

In accordance with one aspect of the present invention, there is provided a method of vending powder. The method includes providing a reservoir for storing powder, having an opening for dispensing the powder; providing a mixer disposed within said reservoir; providing a motion generation device in mechanical communication with the mixer; and providing a controller for selecting a specific volume of the powder to be dispensed. The method further includes, upon receiving user input via the controller selecting the specific volume of the powder, selectively causing the motion generation device to rotate the mixer thereby mixing the powder; and causing the specific volume of powder to be dispensed though the opening.

### BRIEF DESCRIPTION OF DRAWINGS

In the figures, which illustrate by way of example only, embodiments of the present invention:

FIG. 1 is a perspective view of a chalk vending apparatus, exemplary of an embodiment of the present invention;

FIG. 2 is a perspective view of the reservoir of the chalk vending apparatus of FIG. 1 depicting its feeder, mixer, motion generation devices, motion transmission devices and chute;

FIG. 3 is an elevation view partially in cross section of the reservoir of FIG. 2;

FIG. 4 is an elevation view partly in cross section of a reservoir of in one alternative embodiment of a chalk vending apparatus; and

FIG. 5 is an elevation view partly in cross section of another reservoir of in another alternative embodiment of a chalk vending apparatus.

Specific embodiments will be described in detail, by way of example only, with reference to the attached drawings. The same reference label in the drawings identifies the same or similar elements, parts or portions thereof. Persons of skill in the art will readily understand that the drawings are not necessarily drawn to scale.

### DESCRIPTION OF EMBODIMENTS

In the present disclosure, several alternative embodiments of a vending apparatus suitable for use with powder, chalk, or other fine granular materials that may flow freely when shaken or tilted, exemplary of embodiments of the present invention will be described.

In this disclosure, the use of the word “a” or “an” when used herein in conjunction with the term “comprising” may mean “one,” but it is also consistent with the meaning of “one or more,” “at least one” and “one or more than one.” Any element expressed in the singular form also encompasses its plural form. Any element expressed in the plural form also encompasses its singular form. The term “plurality” as used herein means more than one, for example, two or more, three or more, four or more, and the like. Directional terms such as “top”, “bottom”, “upwards”, “downwards”, “vertically” and “laterally” are used for the purpose of providing relative reference only, and are not intended to suggest any limitations on how any article is to be positioned during use, or to be mounted in an assembly or relative to an environment.

The terms “comprising”, “having”, “including”, and “containing”, and grammatical variations thereof, are inclusive or open-ended and do not exclude additional, un-recited elements and/or method steps. The term “consisting essentially of” when used herein in connection with a composition, use or method, denotes that additional elements, method steps or both additional elements and method steps may be present, but that these additions do not materially affect the manner in which the recited composition, method, or use functions. The term “consisting of” when used herein in connection with a composition, use, or method, excludes the presence of additional elements and/or method steps.

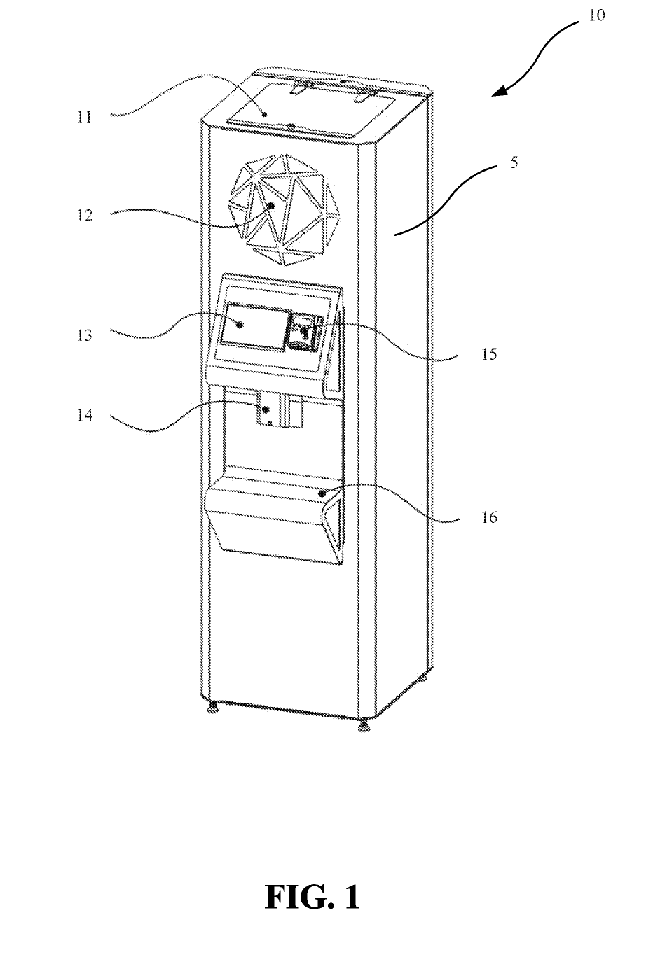

FIG. 1 is a perspective view of a chalk vending apparatus, exemplary of an embodiment of the present invention. As illustrated, a chalk vending apparatus 10 includes a housing 5 containing a reservoir 19 (not shown in FIG. 1). The hosing includes a door 11 located on top the housing 5 providing access to the interior of reservoir 19. A chute 14 in fluid communication with an opening in the reservoir 19 is used to dispense chalk contained therein.

A window 12 formed on one of the walls of housing 5 allows a customer to view whether the chalk or other powder level stored in the reservoir 19 is above a threshold. A tray 16 may be used to place a container to receive powder such as chalk dispensed from the dispensing chute 14.

A controller 13 having a user interface such as a touchscreen or other input-output interface, provides a customer with a means of choosing the quantity of product or powder or chalk in the reservoir 19 desired. Controller 13 may be formed as a touchscreen display or alternately may include individual dedicated buttons for user input and a graphical or character based display. Many alternatives would be apparent to persons of skill in the art.

A payment system 15 coupled to the controller 13 is adapted to receive payment from the customer and provide an indication or a signal that the payment is received. The signal may be provided to the controller 13 directly. The payment system 15 is adapted to use one or more of cash, prepaid cards or tags, credit cards or debit cards, NFC payment methods, cell-phone payment, customer account billing, VIP card, a payment code, online transfer, a bank transfer and the like. Accordingly payment system 15 may be in data communication with remote servers either wirelessly or using a wired communication interface.

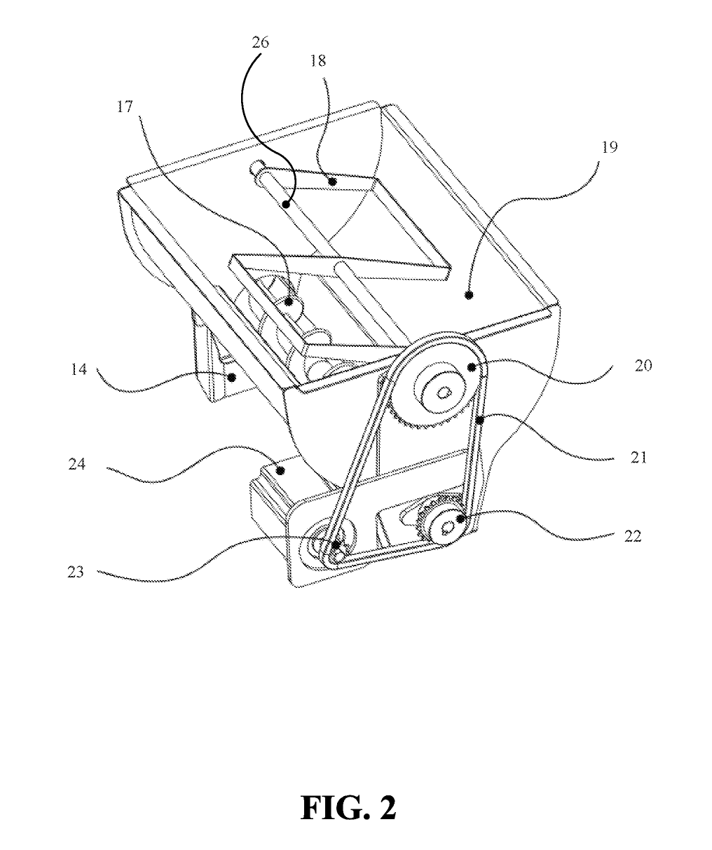

FIG. 2 is a perspective view of the reservoir 19 disposed within housing 5 of powder vending apparatus 10 in one specific exemplary embodiment. As depicted, the apparatus 10 has a feeder 17 and a mixer 18 each disposed within reservoir 19, a motion generation device 24 and motion transmission devices 20, 21, 22, 23.

In embodiments such as that shown in FIG. 2, the motion generation device 24 may be a motor, and the motion transmission devices 20, 21, 22, 23 may respectively be a first pulley, a belt, a second pulley, and a third pulley. In other embodiments, motion transmission devices 20, 21, 22, 23 may respectively be a first gear, a chain, a second gear, and a third gear. Other power transmission mechanisms or drive systems, which may include different combinations of gears or pulleys, belts or chains, or interlocking gears will be known to persons of skill in the art may be used.

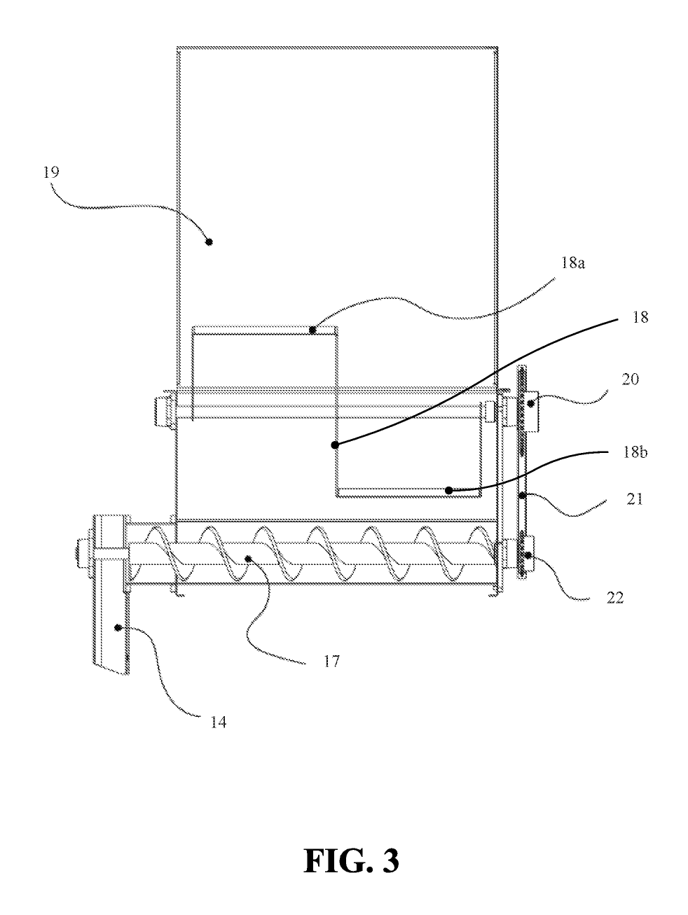

FIG. 3 is an elevation view partially in cross section of the reservoir of FIG. 2. Ad depicted, mixer 18 includes parts 18a, 18b which lie in the same plane, coupled to rod 26. Motion generation device 24 in the belt or chain drive subsystem as depicted in FIG. 2 and FIG. 3, causes rotation of feeder 17 as well as the rotation of by the mixer 18 along the longitudinal axis of its rod 26, thereby mixing chalk in reservoir 19.

In operation, the reservoir 19 is initially filled with chalk for example through door 11 and provided for use by a customer. The customer uses the touch display or alternative equivalent input means of controller 13 to select the quantity of chalk, and pays the corresponding amount using payment system 15.

After payment is completed, the controller 13 receives indication that payment is completed from payment system 15, and thereafter causes chalk to be dispensed through the chute 14. This may be accomplished by signaling the motion generation device 14 (e.g., turning on the motor) so as to cause the feeder 17 to turn and move chalk towards chute 14. Feeder 17 and mixer 18, are actuated through a series of motion transfer devices 20, 21, 22, 23 which themselves actuated by the motion generation device 24.

In some embodiments a valve or some other flow control device (not specifically illustrated) may be disposed between an opening in reservoir 19 and the chute 14 and used to control the flow of chalk out of the reservoir 19.

A customer can then fill his or her own bag or container by placing it under the chute 14. The tray 16 is positioned under the chute 14 may be used to recover excess chalk that was not received within the container. Conveniently, a customer will be able to swipe any chalk left on tray 16 into his or her container.

As noted above, chalk is a non-homogeneous material often having irregular granularity. Such materials are known to be subject to self-compaction inside reservoirs, which makes them difficult to distribute in existing vending machines. The operation of the apparatus 10 whereby as the feeder 17 is turned to move chalk towards chute 14, the motion generation device 24 also rotates mixer 18 along the longitudinal axis of its center rod 26, mixes the chalk in reservoir 19. The mixing or stirring by mixer 18 reverses the self-compaction of chalk or other self-compacting material inside reservoir 19 and permits the chalk to more freely move along its fluid path to the chute 14.

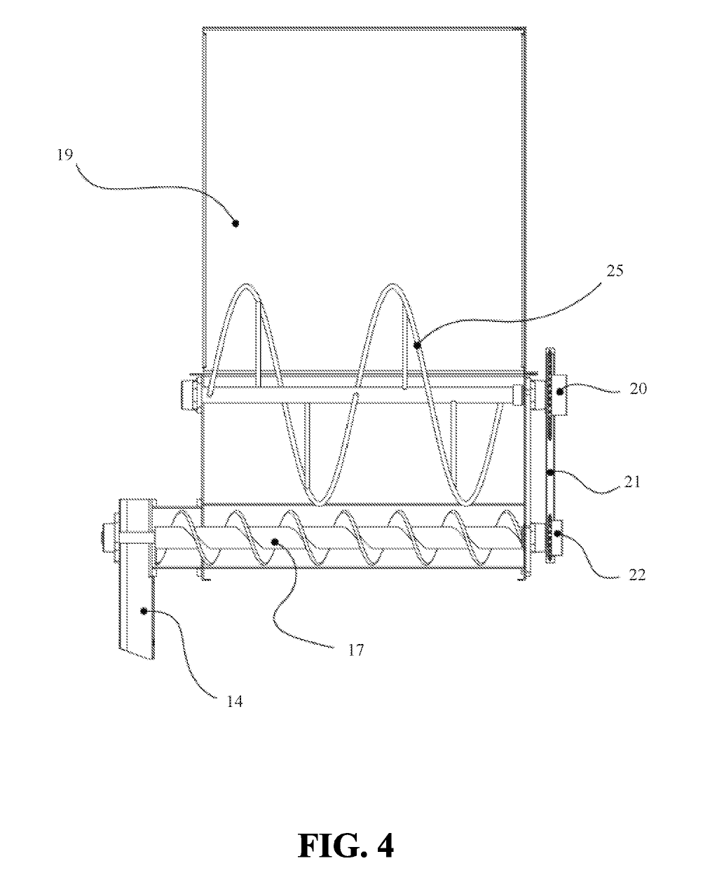

FIG. 4 depicts an alternative embodiment of a mixer 25 inside the reservoir 19. The reservoir 19 is used the store bulk chalk. Inside such reservoir are placed a feeder 17 and the mixer 25. Feeder 17 and mixer 25 are actuated through the series of motion transfer devices 20, 21, 22, 23 themselves actuated by motion generation device 24. Mixer 25 includes an axial center rod coupled to a part having a sinusoidal or helical shape.

Similarly to the earlier embodiment, in embodiments such as that shown in FIG. 4, the motion generation device 24 may be a motor, and the motion transmission devices 20, 21, 22, 23 may respectively be a first pulley, a belt, a second pulley, and a third pulley. In other embodiments, motion transmission devices 20, 21, 22, 23 may respectively be a first gear, a chain, a second gear, and a third gear. Other power transmission mechanisms or drive systems, which may include different combinations of gears or pulleys, belts or chains, or interlocking gears will be known to persons of skill in the art may be used.

Again, as the feeder 17 is turned to move chalk towards chute 14, the motion generation device 24 also rotates mixer 25 along the longitudinal axis of its center rod mixes the chalk in reservoir 19. A different kind of mixing or stirring action is accomplished by the structure of mixer 25 which differs structurally from mixer 18. This again permits the chalk to more freely move along its fluid path to the chute 14.

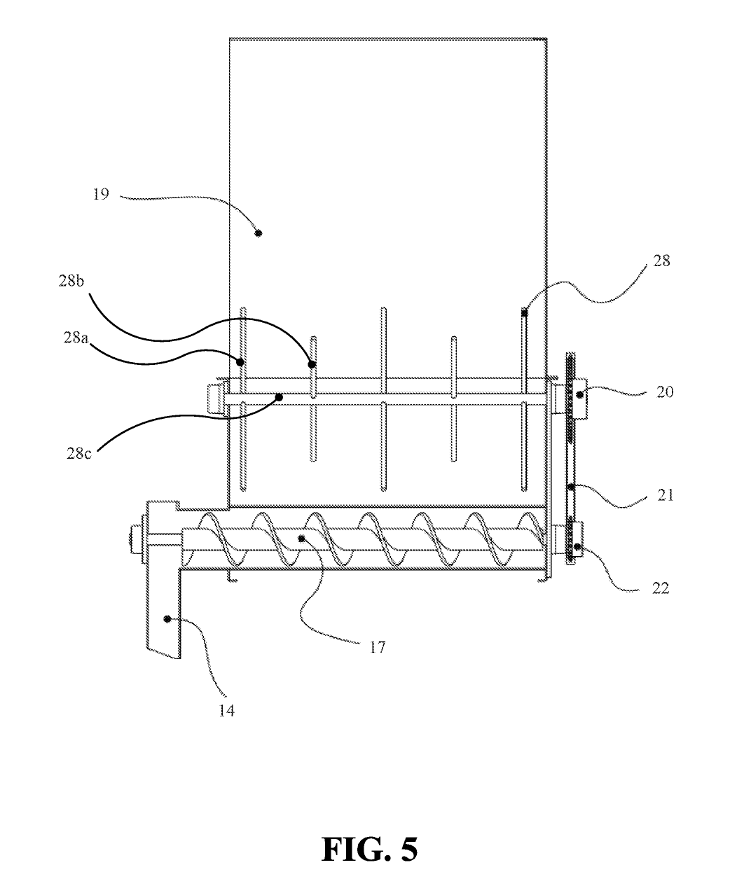

FIG. 5 depicts another alternative embodiment of a mixer 28 inside the reservoir 19 storing bulk chalk. The mixer 28 and the feeder 17 are disposed inside the reservoir 19. Feeder 17 and mixer 28 are actuated through the series of motion transfer devices 20, 21, 22, 23 themselves actuated by motion generation device 24. As depicted in FIG. 5, mixer 28 includes axial center rod 28c having a plurality radially outwardly projecting spokes 28a, 28b for mixing the powered or chalk.

Similarly to the earlier embodiments of FIG. 3 and FIG. 4, in the embodiment shown in FIG. 5, the motion generation device 24 may be a motor, and the motion transmission devices 20, 21, 22, 23 may respectively be a first pulley, a belt, a second pulley, and a third pulley. In other embodiments, motion transmission devices 20, 21, 22, 23 may respectively be a first gear, a chain, a second gear, and a third gear. Other power transmission mechanisms or drive systems, which may include different combinations of gears or pulleys, belts or chains, or interlocking gears will be known to persons of skill in the art may be used.

As the feeder 17 is turned to move chalk towards chute 14, the motion generation device 24 also concurrently rotates mixer 28 along the longitudinal axis of its center rod mixes the chalk in reservoir 19. A different kind of mixing or stirring action is accomplished by the structure of mixer 28 which differs structurally from mixer 18 and mixer 25. This permits the chalk to more freely move along its fluid path to the chute 14.

It should be understood that the feeder and mixer (i.e., feeder 17 and mixer 18 or mixer 25 or mixer 28) may be rotated at the same speed. In alternate embodiments the feeder and mixer may be rotated at different speeds.

In some embodiments the feeder and mixer (i.e., feeder 17 and mixer 18 or mixer 25 or mixer 28) may be rotated concurrently, while in other embodiments the mixer may be rotated first, then followed by rotation of the feeder 17.

In alternate embodiments the feeder and mixer (i.e., feeder 17 and mixer 18 or mixer 25 or mixer 28) may be independently actuated and more than one motor or motion generating device may be employed.

Controller 13 may include a processor, memory and processor executable instructions adapted to control touch screen display or other means of displaying information and other means of getting input from the customer such as keypads, keyboards and the like.

While the present invention has been described with reference to the particular illustrative embodiments, it is to be understood that the invention is not to be limited by the particular details of the embodiments described but only by the appended claims. Persons of skill in the art would understand that the implementation details of the present invention can be modified without departing from the scope of the claims.

What is claimed is:

1. A vending apparatus comprising:
a) a reservoir for storing powder, having an opening for dispensing the powder;
b) a mixer disposed within said reservoir;
c) a motion generation device in mechanical communication with the mixer; and
d) a controller for selecting a specific volume of the powder to be dispensed, the controller operable to selectively cause the motion generation device to rotate the mixer thereby mixing the powder and to cause the specific volume of powder to be dispensed though the opening.

2. The apparatus of claim 1, wherein the powder is bulk chalk.

3. The apparatus of claim 1, comprising a chute in fluid communication with the reservoir through the opening, for dispensing the powder from the reservoir.

4. The apparatus of claim 1, further comprising a payment system coupled to the controller for receiving payment corresponding to the specific volume selected, the controller causing the specific volume of powder to be dispensed in response to the payment system receiving the payment.

5. The apparatus of claim 1, further comprising a feeder disposed inside the reservoir.

6. The apparatus of claim 1, wherein the mixer has an axial center rod coupled to a plurality of parts arranged within the same plane.

7. The apparatus of claim 1, wherein the mixer has an axial center rod having a plurality of radially outwardly projecting spokes.

8. The apparatus of claim 1, wherein the mixer has an axial center rod coupled to a part having one of a sinusoidal and helical shape.

9. The apparatus of claim 5, wherein the motion generation device rotates the feeder to move the specific volume of powder to be dispensed through the chute.

10. The apparatus of claim 9, wherein the motion generation device simultaneously rotates the feeder and the mixer.

11. The apparatus of claim 10, wherein the feeder and the mixer rotate at different speeds.

12. The apparatus of claim 10, wherein the feeder and the mixer rotate at the same speed.

13. The apparatus of claim 1, wherein the controller comprises a user interface allowing a customer to choose the desired chalk quantity to be dispensed from the reservoir.

14. The apparatus of claim 13, wherein the user interface comprises a touchscreen.

15. The apparatus of claim 1, further comprising a housing containing the reservoir inside.

16. The apparatus of claim 15, further comprising a window on a wall of said housing for viewing powder level in the reservoir.

17. The apparatus of claim 16, further comprising a door on said housing providing access into the reservoir.

18. The apparatus of claim 5, further comprising motion transmission devices for transmitting motion from the motion generating device to the mixer and the feeder.

19. The apparatus of claim 18, wherein the motion transmission devices comprise at least one of a gear, a chain, a pulley and a belt.

20. A method of vending powder comprising:
a) providing a reservoir for storing the powder, having an opening for dispensing the powder;
b) providing a mixer disposed within said reservoir;
c) providing a motion generation device in mechanical communication with the mixer; and
d) providing a controller for selecting a specific volume of the powder to be dispensed, wherein
i) upon receiving user input via the controller selecting the specific volume of the powder, selectively causing the motion generation device to rotate the mixer thereby mixing the powder; and
ii) causing the specific volume of powder to be dispensed though the opening.
